(19) United States
(12) Patent Application Publication
(10) Pub. No.: US 2019/0038184 A1
(43) Pub. Date: Feb. 7, 2019

(54) METHOD AND AN ELECTRONIC DEVICE FOR TRACKING A USER ACTIVITY

(71) Applicant: SAMSUNG ELECTRONICS CO., LTD., Suwon-si (KR)

(72) Inventors: Navin NARASIMHAN, Bangalore (IN); Tuhin CHAKRABORTY, Bangalore (IN); Kiran Prasanth RAJAN, Bangalore (IN); Vinayak PANCHOL, Bangalore (IN); Hema ARAVINDAN, Bangalore (IN); Khaidem Suman SINGHA, Bangalore (IN); Parmarth Vawa Sarathi RAI, Bangalore (IN); Saumitri CHOUDHURY, Bangalore (IN); Hallah ZORINSANG, Bangalore (IN)

(73) Assignee: SAMSUNG ELECTRONICS CO., LTD., Suwon-si (KR)

(21) Appl. No.: 16/054,572

(22) Filed: Aug. 3, 2018

(30) Foreign Application Priority Data
Aug. 4, 2017 (IN) 201741004751

(51) Int. Cl. A61B 5/11; G06F 3/0488; G06Q 10/10; G06F 1/16; G06F 3/0484

(52) U.S. Cl. CPC A61B 5/1118; G06F 3/04883; A61B 5/1102; G06F 3/04842; G06Q 10/109; G06F 1/163; G06F 3/04847; A61B 5/1112

(57) ABSTRACT

An electronic device and a method for tracking a user activity using the electronic device are provided. The electronic devices includes a display, a sensor unit, and a processor electrically connected to the display and the sensor unit, wherein the processor is configured to control the display to display a user interface (UI); to receive an input from a user, via the UI, to display one or more activities associated with the user, to control the sensor unit to extract context information of the one or more activities associated with the user; and to control the display to display an indication of the one or more activities associated with the user in any of various forms on the UI.

### CROSS-REFERENCE TO RELATED APPLICATION(S)

This application is based on and claims priority under 35 U.S.C. § 119 to Indian patent application No. 201741004751 filed on Aug. 4, 2017, in the Indian patent Office, the disclosure of which is incorporated by reference herein in its entirety.

### BACKGROUND

### 1. Field

Apparatuses and methods consistent with example embodiments relate to an electronic device which senses and displays information about user activity and a method therefor.

### 2. Description of the Related Art

Current-generation electronic devices can perform various functions in an integrated manner. For instance, electronic devices such as smartphones and other portable terminals provide users with more convenience along with better performance. The functions that are offered by the electronic devices are provided using one or more sensors within the devices. Such sensors can gather information related to another electronic device, an environment surrounding outside the electronic device, and the user.

Electronic devices can include one or more sensors and provide various services using information gathered through the sensors. Current smart devices may include sensors and software which can track a user's activities, but their representation of the information they obtain is not coherent. Existing time management solutions include planners and activity history trackers.

The existing technology does not reflect the overlap and multi-tasking of activities of real life. One conventional technology provides a smart watch with a circular graphical representation with which a user can only track movement, exercise and standing, from day to day in a quantitative measure. The technology provides a graphical representation comparing durations of only these three specific activities.

Another existing technology provides a smart device comprising a circular rotating bezel used for navigation. The smart device also includes smart device sensors for performing activity tracking, and may include a scheduler/planner. However, current smart devices provide only limited options for tracking a users' activities and planning out the activities of a day.

In view of the foregoing, there is a need to seamlessly track not only past activities of but also current activities and future plans, in order to provide a holistic understanding of life events and a plan for the future. Further, there is a need for a method of sending a user's activities and providing both qualitative and quantitative measures of the activities to the user in order to support more efficient time management. There is also Further, a need for a device that allows a user to go back and review past activities as well as to perform functions pertinent to future activities. There is a need for a smart device that can analyze past activities, offer, in the present, intelligent recommendations for the future based on the past activities, and allow for automatic readjustment of a scheduler with the user's consent. Finally, there is a need for a smart device that can track and display multiple activities being performed by the user at the same time.

### SUMMARY

Various example embodiments may provide a method and an electronic device for tracking a user activity. According to one example embodiment, an electronic device comprises a display, a sensor unit and at least one processor electrically connected to the display and the sensor unit, wherein the processor is configured to control the display to display a user interface (UI), receive an input from a user, via the UI, to display an activity associated with the user, control the sensor unit to extract context information of the activity associated with the user, and control the display to display, on the UI, an indication of the activity associated with the user.

According to another example embodiment, a method comprises displaying a user interface (UI), receiving an input from a user, via the UI, to display an activity associated with the user, extracting, by a sensor unit, context information of the activity associated with the user; and displaying, on the UI, an indication of the activity associated with the user.

According to another example embodiment, a wearable electronic device comprises a display, a sensor unit configured to obtain information about a user, at least one processor electrically connected to the display and the sensor unit, wherein the processor is configured to control the display to display a user interface (UI) comprising information regarding a plurality of activities of the user comprising a past activity, a present activity, and a future activity, control the sensor unit and thereby obtain sensed information about the user, control a display of at least one of the past activity, the present activity, and the future activity based on the sensed information about the user.

### BRIEF DESCRIPTION OF THE ACCOMPANYING DRAWINGS

These and/or other example objects, features and advantages will become apparent to those skilled in the art from the following description of example embodiments and the accompanying drawings in which:

FIG. 1 is a schematic diagram illustrating an exemplary representation of one or more activities of a user being displayed on a user interface (UI) of an electronic device, according to an example embodiment. The UI may be circular, or may be any other shape, as would be understood by one of skill in the art.

FIG. 2A is a flow chart illustrating an exemplary method of indicating one or more user activities on a display of an electronic device, according to an example embodiment.

FIG. 2B is a diagram illustrating a method for displaying detailed information of a selected activity if a specific activity is selected by a user.

FIG. 2C is a flowchart illustrating a method for recommending an activity to a user.

FIG. 3A is a block diagram illustrating an exemplary electronic device as shown in FIG. 1 having one or more functional components, according to an example embodiment.

FIG. 3B illustrates an exploded view of an activity tracking module as such those shown in FIG. 3A, according to an example embodiment.

FIG. 4A is a schematic diagram illustrating a possible display in a scenario in which a user scans through future activities, according to an example embodiment.

FIG. 4B is a schematic diagram illustrating a possible display in a scenario in which a user scans through past activities, according to an example embodiment.

FIG. 5 is a schematic diagram illustrating a scenario in which a user performs a shake gesture to toggle between a time mode and a data mode to view details of an activity associated with the user on the electronic device, according to an example embodiment.

FIGS. 6A-6B are schematic diagrams illustrating a scenario in which a user scans through his past activities, according to an example embodiment.

FIGS. 7A-7B are schematic diagrams illustrating a scenario in which a user scans through one or more past activities on an electronic device, according to another example embodiment.

FIGS. 8A-8B are schematic diagrams illustrating a scenario in which a user scans through one or more past activities on an electronic device, according to another example embodiment.

FIGS. 9A-9C are schematic diagrams illustrating a scenario in which a user scans through past activities, according to another example embodiment.

FIGS. 10A-10B are schematic diagrams illustrating a scenario in which a user manages activities, according to an example embodiment.

FIGS. 11A-11B are schematic diagrams illustrating a scenario in which a user creates a new activity to be performed in future, according to an example embodiment.

FIG. 12 is a flowchart illustrating an exemplary method of displaying one or more sub-activities associated with a main activity, according to an example embodiment.

### DETAILED DESCRIPTION

One or more example embodiments may provide a method for tracking a user activity using an electronic device. In the following detailed description of example embodiments, reference is made to the accompanying drawings that form a part hereof, and in which are illustrated specific example embodiments. These example embodiments are described in sufficient detail to enable those skilled in the art to practice them, and it is to be understood that other example embodiments may be utilized and that changes may be made. The following detailed description is, therefore, not to be taken in a limiting sense, and the scope of the present application is defined only by the appended claims.

The specification may refer to “an”, “one” or “some” embodiment(s) in several locations. This does not necessarily imply that each such reference is to the same embodiment(s), or that the feature only applies to a single embodiment. Single features of different embodiments may also be combined to provide other embodiments.

As used herein, the singular forms “a”, “an” and “the” are intended to include the plural forms as well, unless expressly stated otherwise. It will be further understood that the terms “includes”, “comprises”, “including” and/or “comprising” when used in this specification, specify the presence of stated features, integers, steps, operations, elements and/or components, but do not preclude the presence or addition of one or more other features, integers, steps, operations, elements, components, and/or groups thereof. As used herein, the term “and/or” includes any and all combinations and arrangements of one or more of the associated listed items.

Unless otherwise defined, all terms (including technical and scientific terms) used herein have the same meaning as commonly understood by one of ordinary skill in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The example embodiments herein and the various features and advantages details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein can be practiced and to further enable those of skill in the art to practice the embodiments herein. Accordingly, the examples should not be construed as limiting the scope of the example embodiments herein.

Some example embodiments are directed to various activities performed by a user on the electronic device. For example, the electronic device may determine that the user typically calls her parents on Friday night, listens to certain types of music while commuting to work, listens/reads/watches the news while commuting home, plays certain types of games at certain locations (e.g., dentist/doctor office), browses the Internet and has other such habits. The electronic device identifies these activities and generates a recommendation for the at least one activity that can be performed by the user.

An example electronic device displays the tracked one or more user activities or events or actions performed by the user on a display of the electronic device. The one or more activities of the user may be displayed in a circular time based demographic format giving both quantitative and qualitative measure of the activity. The qualitative measure represents the activity in time, whereas the quantitative measure represents other data of the activity. One or more example embodiments may also allow the user to scan past and future activities by use of a gesture displayed in a clockwise and anti-clockwise direction on the circular user interface (UI) of the electronic device. The gesture can be a circular swipe or a rotate gesture. In one example embodiment, the circular UI of the electronic device may display the present time, date, time of an activity, an icon representing the activity, a name of the activity, a duration of activity, a pointer pointing at a color representing the activity in the multi-color activity time table chart, and a current time indicator for representing the current time on the electronic device.

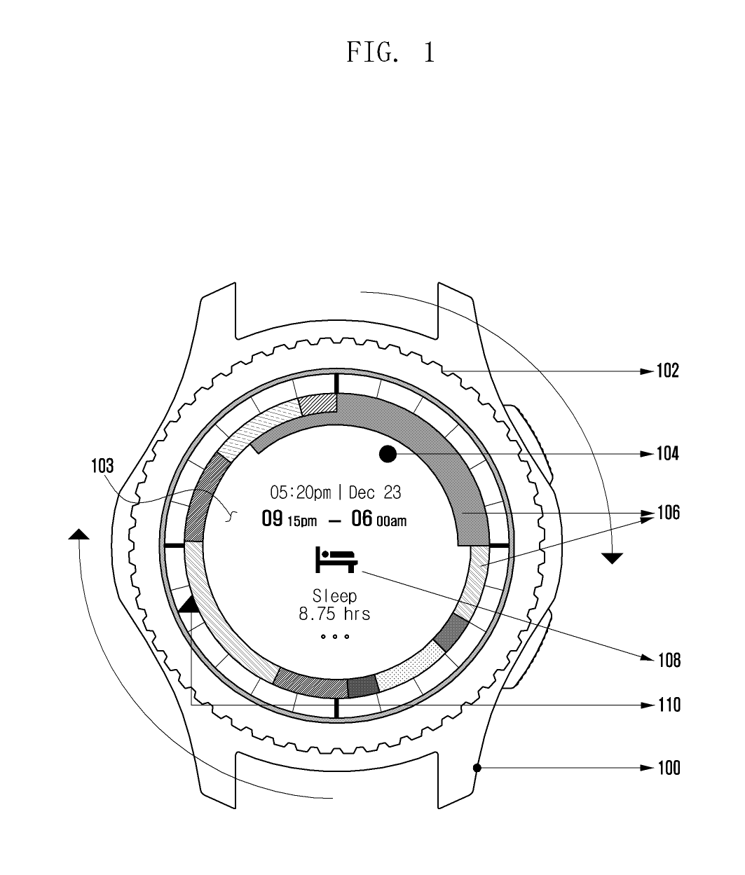

FIG. 1 is a schematic diagram illustrating an exemplary representation of one or more activities of a user being displayed on a circular user interface (UI) of an electronic device, according to an example embodiment. As shown in FIG. 1, the electronic device 100 corresponds to at least one of a smartphone, a smartwatch, a mobile device, a personal computer, a tablet, a wearable device, and a dual screen mobile device. According to various example embodiments, the electronic device 100 corresponds to a smartwatch which comprises a circular rotating bezel 102, and a display 103. The circular rotating bezel 102 is used to show past and future activities in a circular time based format with current time as a conceptual pivot point. For example, if the user rotates the bezel 102 in an anti-clockwise direction, then the activities performed by the user in the past are displayed on the display 103. On the contrary, if the user rotates the bezel 102 in a clockwise direction, then the activities to be performed by the user in the future are displayed on the display 103. According to various example embodiments, the electronic device 100 control the display 103 to display a circular UI which comprises an activity pointer 104, a circular activity time table chart 106, a center information space 108, and a current time indicator 110. In one example embodiment, each activity is indicated by a different color arc in the circular activity time table chart 106, wherein the length of arc defines the duration of the activity. The current time indicator 110 indicates a current time and follows a 24-hour time format. The activity pointer 104 highlights the activity selected by the user on the circular activity time table chart 106. In one example embodiment, the indication of activity displayed on the circular UI may be an icon, a color, an image or a combination thereof.

An exemplary indication of a plurality of activities with different colors or different patterns of arcs are shown in FIG. 1. According to an example embodiment, the electronic device can variously change the shape and/or information of the arcs being currently performed. For example, the electronic device may semi-permanently display the activity being currently performed, and then may display the activity with a deeper color as completion is approached. Now, consider that the user has selected a first arc on the circular activity time table chart 106 to view the activity performed at that time. In response, the electronic device 100 controls the display 103 to display details of the activity regarding to the first arc on the center information space 108. As can be seen, the electronic device 100 displays information such as the fact that the activity performed was a sleep activity for a duration of 8.75 hours from 9.15 pm to 6.00 am on December 23. Since, the sleep activity extended into the next day, the activity may be represented as a bigger arc than the arcs of other activities to indicate overlap in time. In one example embodiment, the electronic device comprises one or more sensors to track one or more activities of the user.

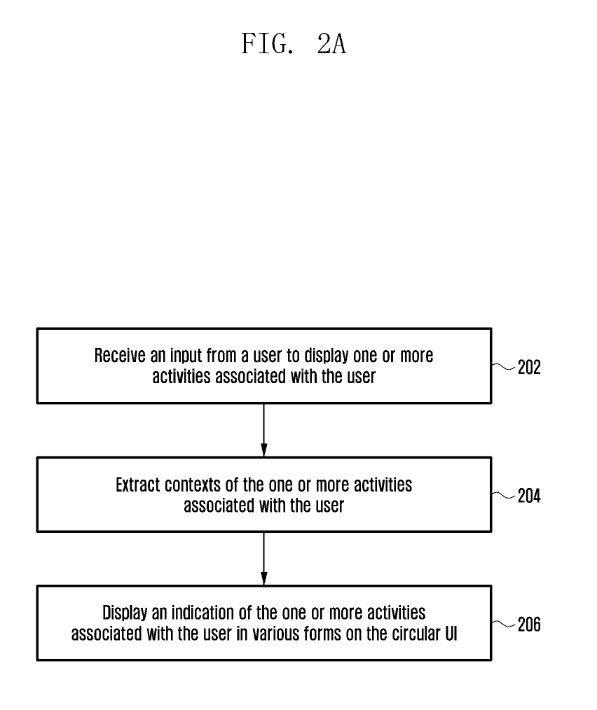

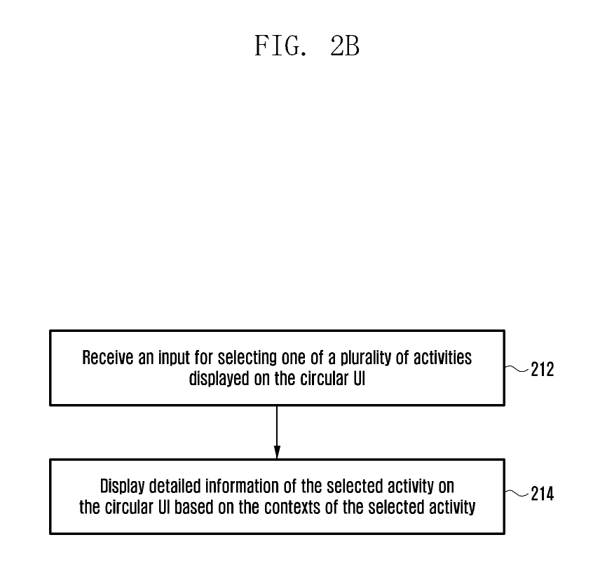

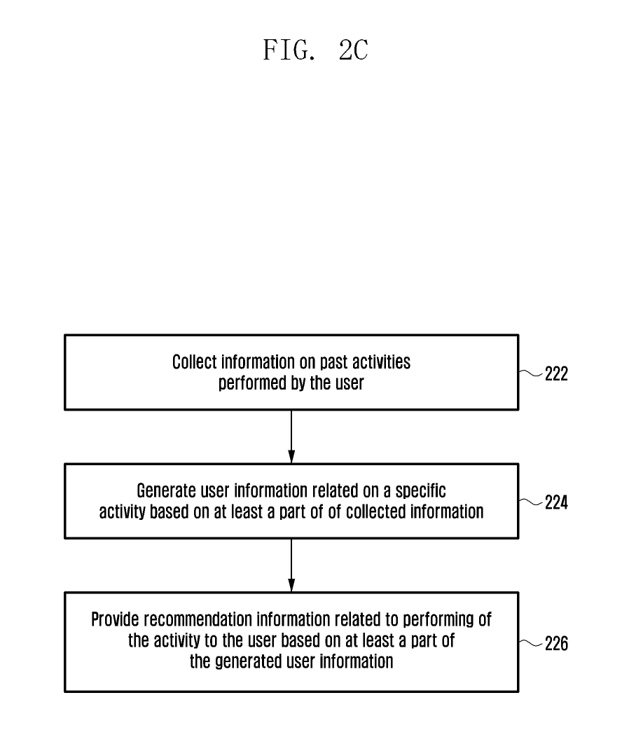

FIG. 2A is a flow chart illustrating an exemplary method of indicating one or more activities of a user on a display of an electronic device, according to an example embodiment. At step 202, the electronic device receives an input from the user to display, by using a circular user interface (UI), one or more activities associated with the user.

The input may include, for example, the execution of an application for displaying the circular UI and/or an input comprising a user gesture predetermined to control the display of the circular UI.

According to various example embodiments, the electronic device may collect activities performed and to be performed “today” in response to the input. For example, the electronic device may collect activities already performed today and activities to be performed today based on the time when the input is received. According to various example embodiments, the performed activities may be collected based on at least a portion of an activity item generated by the user and sensing information of the electronic device. For example, the electronic device may sense user movement using a sensor unit, and even if there is no user input, a period in which the movement is sensed may be collected as “a travel activity”.

According to various example embodiments, the activities to be performed may be collected based on the activity item generated by the user or the pre-performed activities. For example, the electronic device may determine performance of “a sleep activity” in a period from 9.15 pm to 6.00 am as a user's life pattern, and even in a case in which there has been no user input, the electronic device may generate a sleep activity.

According to various example embodiments, the input may include date information selected by the user. For example, the user may include the date information in the input to see activities performed “yesterday” or to be performed “tomorrow”.

In response to the input, the electronic device, at step 204, extracts contexts of the one or more activities associated with the user, wherein the contexts of the one or more activities comprises at least one of time information of a time during which the activity was performed or is to be performed, location information of a location at which the activity was performed or is to be performed, a type of activity, a duration of the activity, a status of the activity, and behavior and habitual information associated with the user. According to an example embodiment, the electronic device may store information of the time at which and/or information of the location at which the activity is to be performed based on the user's input. In one example embodiment, the contexts associated with one or more activities are obtained by using a sensor unit. For example, the electronic device may confirm the time and/or the location of an activity that has been performed using the sensor unit. As another example, the electronic device may measure a user's state change (e.g., heart rate or brainwave change) occurring during performance of a specific activity, and may determine the behavior and habitual information associated with the user occurring during performance of the activity.

In this example embodiment, the sensor unit comprises, but is not limited to, a biometric sensor, a physiological sensor, an accelerometer, a gyroscope, a Polysomnography sensor, a ballistocardiography sensor, a motion sensor, a global positioning system (GPS) receiver, and proximity sensors. In some example embodiments, the sensor unit is adapted to monitor at least one of cardiac activity, hemodynamics, respiratory function, neurological function, body temperature, sleep pattern, stress, toxicity, and one or more activities of the user. The one or more activities may correspond to physical activity and/or an activity performed by the user on the electronic device.

At step 206, an indication of the one or more activities associated with the user are displayed in any of various forms on the circular UI of the electronic device based on the extracted contexts. For example, the one or more activities associated with the user may be displayed in a circular activity time table chart (e.g., a circular activity time table chart 106 of FIG. 1) of the circular UI based on the information of the time at which the activity was performed or is to be performed. As another example, one or more activities associated with the user may be differentiated and displayed in different colors or different patterns in accordance with the type of activity.

In one example embodiment, each of a plurality of activities is identified using a different color or different pattern of arcs along with qualitative and quantitative measure of the activity. The qualitative measure represents a time of the, whereas the quantitative measure represents data of the activity. The representation of the qualitative and quantitative measures of an activity are discussed in the following sections in detail.

FIG. 2B is a diagram illustrating a method for displaying detailed information of a selected activity if a specific activity is selected by a user.

At step 212, the electronic device may receive an input for selecting one of a plurality of activities displayed on the circular user interface (UI). For example, a user may select an activity via a display (e.g., display 103 of FIG. 1) by using a touch input, or may select an activity via a circular rotating bezel (e.g. circular rotating bezel 102 of FIG. 1) using a pivot input.

Further, according to a certain example embodiment, the electronic device may receive an input for designating a specific time, and may select an activity performed or to be performed at the specific time. For example, if “8 pm” is received from the user as an input, the electronic device may select the activity corresponding to “8 pm”. The specific time may be a past time or a future time with respect to the time at which when the input is received.

At step 214, the electronic device may display detailed information of the selected activity on the circular UI based on the contexts of the selected activity.

For example, the electronic device may display on the circular UI the selected activity and the time and location at which the selected activity was performed. As another example, the electronic device may display health information or work information in accordance with the type of the selected activity. For example, if the activity type is related to physical exercise, the user's current health information may be displayed to guide the user not to undertake unreasonable exercise. As another example, if the activity type is related to a user's business, a completion rate of the selected business or an average time for completing a specific work may be displayed to improve user's work efficiency.

According to another example embodiment, the user may select the activity being currently performed. In this case, the electronic device may display, on the circular UI, information related to the activity being currently performed. For example, if the user performs a physical activity (e.g., exercise), the electronic device may confirm one of user's brainwave or fatigue using the sensor unit. The electronic device may provide the confirmed brainwave or fatigue to the user, so that the user can recognize the change in accordance with the activity.

FIG. 2C is a flowchart illustrating a method for recommending an activity to a user.

At step 222, the electronic device may collect information on past activities performed by the user. According to an example embodiment, the electronic device may classify the activities by types (or categories), and then may collect information in accordance with the classified types (or categories). For example, the electronic device may classify a physical activity (e.g., exercise) as one item, and may collect the information on the physical activity.

At step 224, the electronic device may generate user information related to a specific activity based on at least a portion of the collected information.

According to one example embodiment, the user information related to the specific activity may include at least one of a statistical value of the activity or user's health information related to performing of the activity. For example, the electronic device may generate as user information the statistical value of the time consumed by the user to perform the activity (e.g., exercise) every day, every week, or every month. As another example, the electronic device may divide the activity into detailed items (e.g., workout and weights), and may generate as the user information the user's health information (e.g., fatigue) measured when the statistical values for the detailed items are generated or the detailed items are performed using the sensor unit.

At step 226, the electronic device may provide, to the user, recommendation information related to performance of the activity, based on at least a part of the generated user information.

According to an example embodiment, the recommendation information may be related to generation, change, or deletion of the activity. For example, the electronic device may recommend peformance of the activity based on the activity statistical value or the user's health information. According to an example embodiment, the electronic device may check whether there is open time in the user's schedule, and may provide to the user recommendation information of the activity (e.g., exercise) that can be performed during the open time. Further, in a certain example embodiment, the electronic device may confirm the activity scheduled to be performed, and may provide to the user the recommendation information to add a performance time. Further, in a certain example embodiment, the electronic device may guide deletion of the activity scheduled to be performed based on the user's health information.

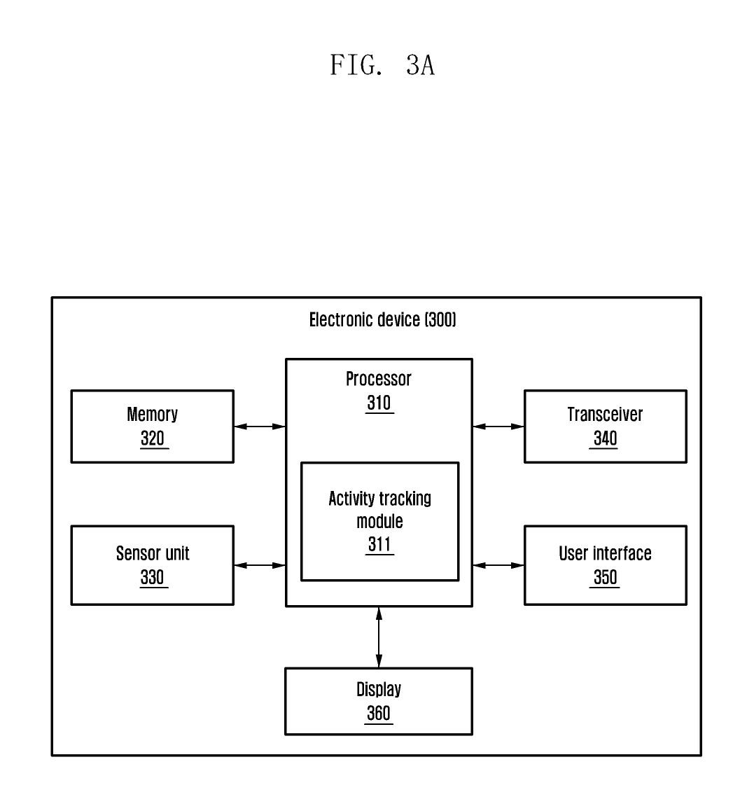

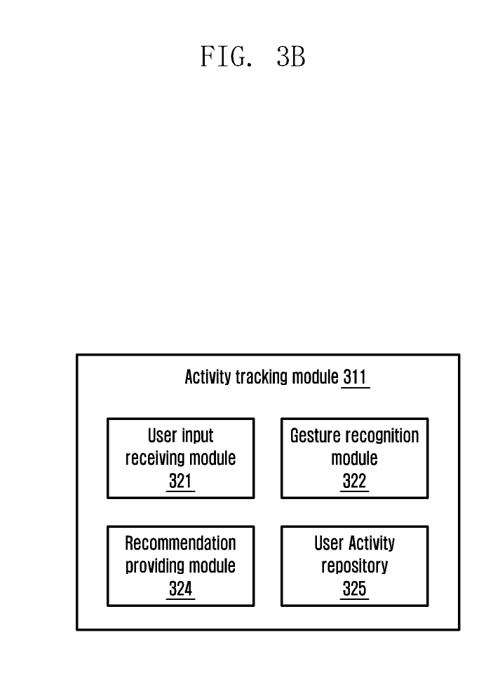

FIG. 3A is a block diagram illustrating an exemplary electronic device as shown in FIG. 1 having one or more functional components, according to one embodiment. As shown in FIG. 3A, the electronic device 300 comprises a processor 310, a transceiver 340, a memory 320, user interface 350 and a display 360. The electronic device 300 may comprise additional components such as network interface, battery, camera etc., which are not shown for the sake of brevity.

The processor 310 may include one or more data processing circuits, such as a general purpose and/or special purpose processor (e.g., microprocessor and/or digital signal processor). The processor 310 is configured to execute one or more functions for the requests received from the activity tracking module 311. The transceiver 340 is adapted for establishing communication with a network of plurality of other mobile electronic terminals. The processor 310 is configured to receive information from the memory 320 and execute one or more functions accordingly. The activity tracking module 311 is adapted for tracking one or more activities associated with a user. The activity tracking module 311 may be configured to control one or more sensors, and other modules as shown in FIG. 3B for tracking one or more user activities. The functions of the activity tracking module 311 are explained in detail in the following sections.

The sensor unit 330 is adapted for sensing one or more activities of the user. In one example embodiment, the sensor unit 330 comprises at least one of a biometric sensor, a physiological sensor, an accelerometer, a gyroscope, a polysomnography sensor, a ballistocardiography sensor, a motion sensor, a global positioning system (GPS) receiver, and proximity sensors. In one example embodiment, the sensor unit 330 is adapted for monitoring at least one of cardiac activity, hemodynamics, respiratory function, neurological function, body temperature, sleep, stress, toxicity and one or more activities of the user, wherein the one or more activities comprises at least one of physical activity and an activity performed on the electronic device by the user. The sensed one or more activities are sent to the display 360. The display 360 is responsible for displaying the sensed activity on a display of the electronic device.

FIG. 3B illustrates an exploded view of an activity tracking module such as those shown in FIG. 3A, according to one example embodiment. As shown in FIG. 3B, the activity tracking module 311 comprises of a user input receiving module 321, a gesture recognition module 322, a recommendation providing module 324, and a user activity repository 325. These modules work in conjunction with each other to track one or more activities of the user.

The user input receiving module 321 is adapted for receiving one or more inputs from the user. The input may comprise any type of gesture including, but not limited to, a touch gesture, a tap gesture, a long press gesture, a short press gesture, a swipe gesture, and a circular rotation gesture on a display of the electronic device to view one or more activities of the user. In some example embodiments, the user may input a circular swipe on the display to toggle between past and future days to view the one or more activities of the user. The gesture recognition module 322 is adapted for recognizing one or more gestures received from the user input receiving module 321. The gesture recognition module 322 identifies the one or more gestures and performs corresponding actions with the help of the processor.

Further, the sensed activity gets stored in a user activity repository 325. The user activity repository 325 is capable of storing past activities as well as real time and future activities of the user so as to enable the recommendation providing module 324 to provide feedback or suggestions to the user. The recommendation providing module 324 is adapted for providing one or more suggestions associated with an activity of the user to improve his life style. For example, the recommendation providing module 324 keeps a track of a user's workout activity timings and may suggest the user to do more workout to burn extra calories to stay fit. In one example embodiment, the recommendation can be provided as an alert, or as any type of visual representation on the display of the electronic device.

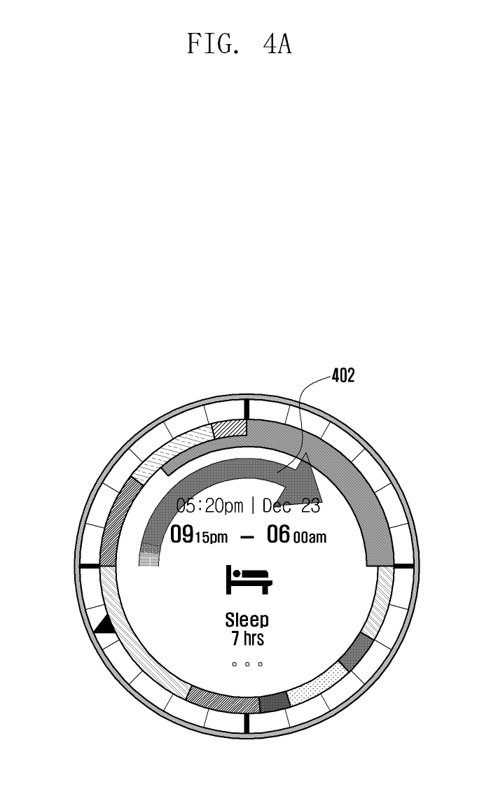

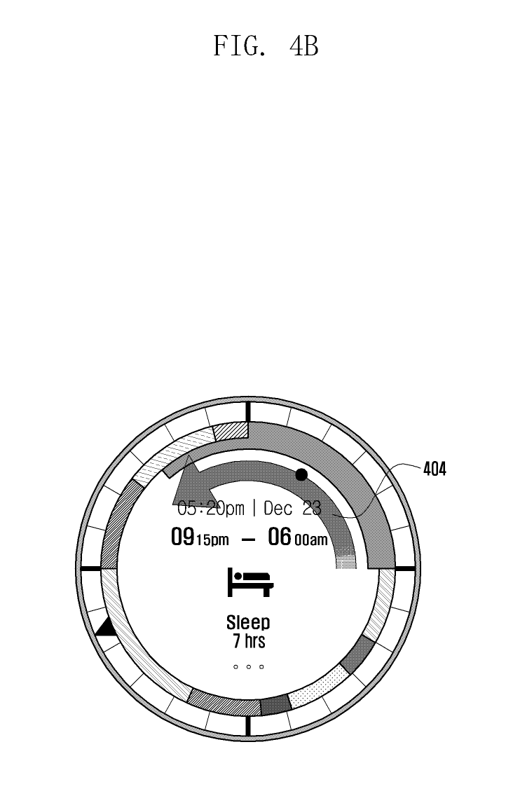

FIG. 4 is a schematic diagram illustrating a scenario in which a user scans through past and future activities performed in a day, according to one example embodiment. According to this example embodiment, consider that a user is viewing a sleep activity on a circular activity time table chart (e.g., a circular activity time table chart 106 of FIG. 1) of the electronic device(e.g., electronic device 100 of FIG. 1). If the user inputs a circular swipe gesture or rotates the bezel in a clockwise direction 402 on the circular UI, the user is then able to view one or more upcoming or scheduled activities. In this case, if the user inputs a circular swipe gesture or rotates the bezel in an anti-clockwise direction 404 on the UI, the user is able to view one or more past activities. The same is illustrated in FIG. 4.

According to various example embodiments, the electronic device may provide a radar scanning function. If execution of the radar scanning function is requested, the electronic device may quickly rotate a current time indicator (e.g., a current time indicator 110 of FIG. 1) in a clockwise direction 402, and may display on the circular UI information of activities indicated by the rotating current time indicator. Accordingly, the user can quickly scan the information of the activities scheduled to be performed. According to another example embodiment of the radar scanning function, the electronic device may quickly rotate the current time indicator in an anti-clockwise direction 404, and may display on the circular UI the information of the activities indicated by the rotating current time indicator. That is, information of the activities performed in the past can be quickly scanned.

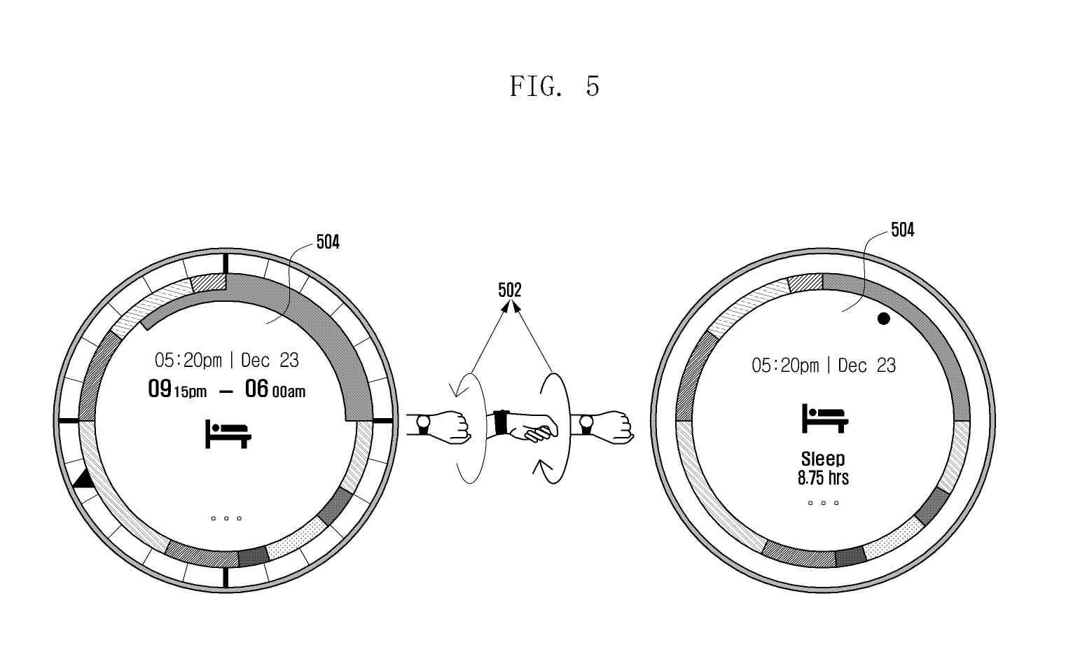

FIG. 5 is a schematic diagram illustrating a scenario in which a user performs a shake gesture to toggle between a time mode and a data mode to view details of an activity associated with user on the electronic device, according to an example embodiment. According to FIG. 5, the electronic device is in a time mode and indicating a sleep activity of the user in the time mode. As shown in first diagram of FIG. 5, a circular UI 504 of the electronic device displays information about the sleep activity in the time mode. Particularly, the circular UI 504 displays that the sleep activity lasted from 9.15 pm to 6.00 am.

In that state, user may perform a shake gesture 502 as shown in FIG. 5 to toggle from the time mode to the data mode. Upon toggling, the circular UI 504 displays only data associated with the sleep activity. That means, the duration of sleep activity of 8.75 hours is displayed on the UI of the electronic device. The same is illustrated in second diagram of FIG. 5.

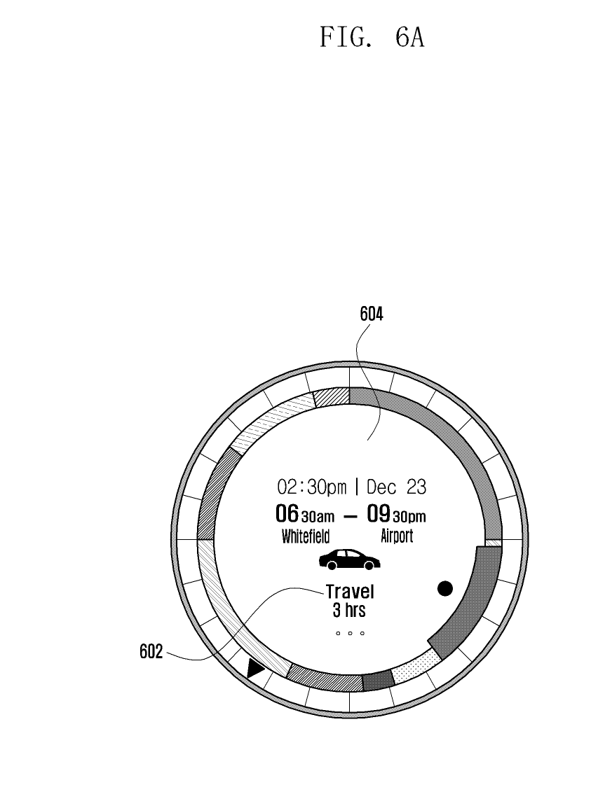

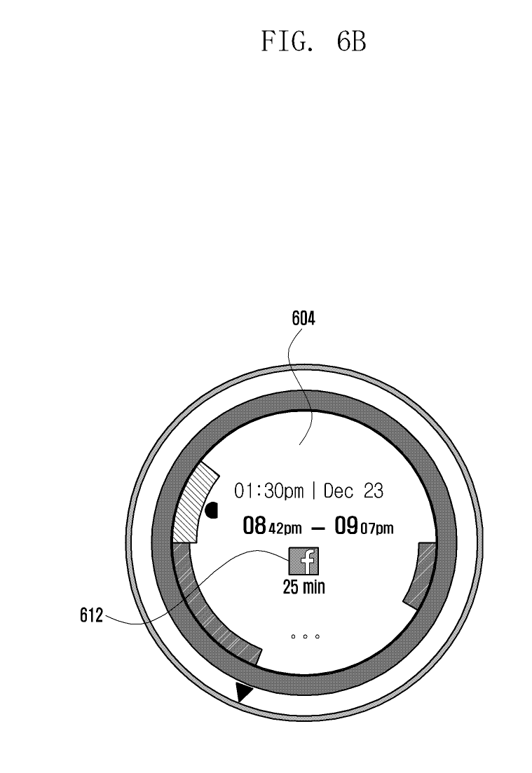

FIG. 6A-6B are schematic diagrams illustrating a scenario in which a user scans through his past activities, according to an example embodiment. In this example, the user was travelling to the airport for a duration of 3 hours from 6.30 pm to 9.30 pm to catch the flight on a first day. The electronic device records the activity as travelling 602 and indicates the activity on a circular UI 604 of the electronic device. The same is illustrated in FIG. 6A. The next day, the user scans through his past activities on the electronic device. At this stage, the user may be interested to know about his performance while travelling to the airport. So, the user requests a display of a sub-activity of the activity ‘travelling’. In response to the user request, the electronic device displays a sub-activity such as ‘browsing a Facebook application 612’. Additionally, the electronic device displays the duration of time (ex: 25 min) during which the user was using the Facebook application on the circular UI. The same is illustrated in FIG. 6B.

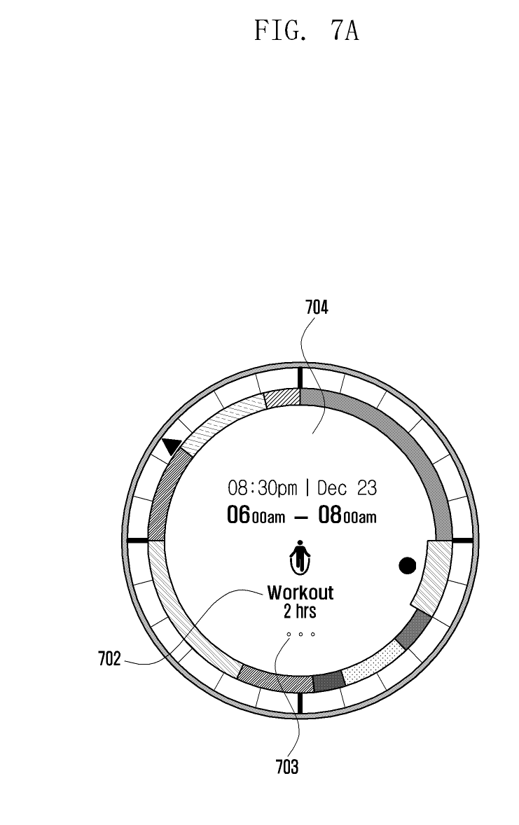

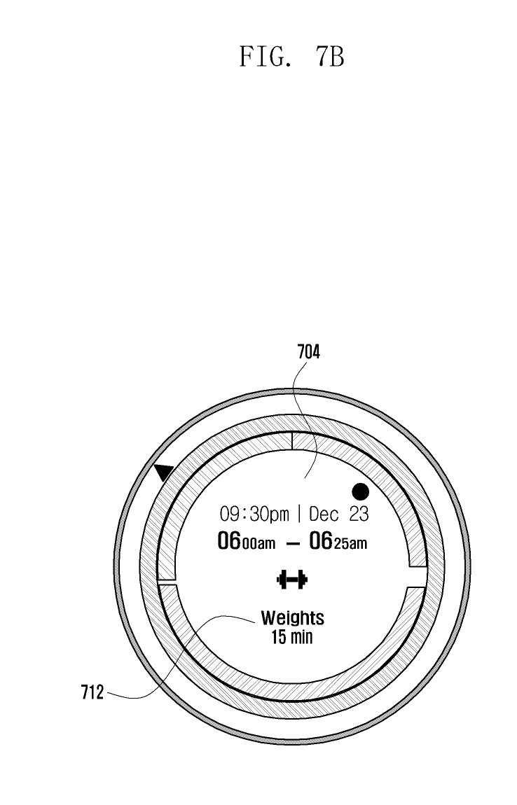

FIGS. 7A-7B are schematic diagrams illustrating a scenario in which a user scans through one or more past activities on an electronic device, according to another example embodiment. In this example embodiment, consider that the user is in a process of exercising at a gym. The user is accustomed to performing various exercises for a duration of 2 hours every day. In one example embodiment, the user may need to calculate the distribution of various exercises that he has performed during a day. Therefore, the user rotates bezel to view the activity, ‘workout 702’ on a circular activity time table chart. As can be seen, the activity ‘workout 702’ is indicated on a circular UI 704 of the electronic device and the activity related information is displayed on a center information space of the circular UI 704. Thus, from the display, the user infers that the he has performed the activity, ‘workout 702’ for a duration of 2 hours from 6.00 am to 8.00 am on December 23. The same is illustrated in FIG. 7A. The user may desire to calculate a distribution of various exercises that he has performed during the time period from 6.00 am to 8.00 am. The user then requests a display of a sub-activity of the activity ‘workout 702’. The electronic device, upon receiving the user request, identifies that the user is interested in viewing a sub-activity associated with a main activity. In this example embodiment, the electronic device considers the main activity as ‘workout’ and considers different exercises performed by the user to be sub-activities.

The electronic device displays different exercises performed by the user for duration of 2 hours from 6.00 am to 8.00 am on December 23. The same is illustrated in FIG. 7B. As shown in FIG. 7B, the user can see that he performed the ‘weights 712’ sub-activity for a duration of 15 mins from 6.00 am to 6.15 am. It is to be noted that the ‘weights 712’ sub-activity is indicated by an icon of a weight. In one example embodiment, an activity performed in parallel to the main activity may be displayed when the user selects one of the three dots 703 as shown in FIG. 7A. Thus, a user can go back in time and view precisely calibrated information of his workout.

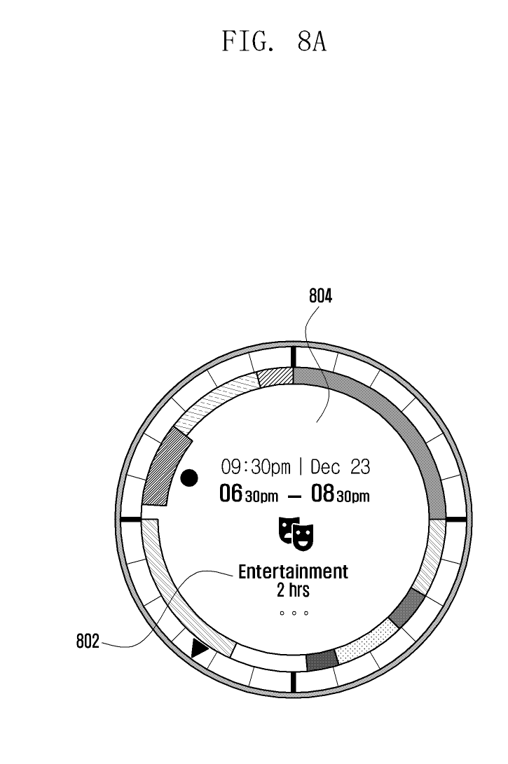

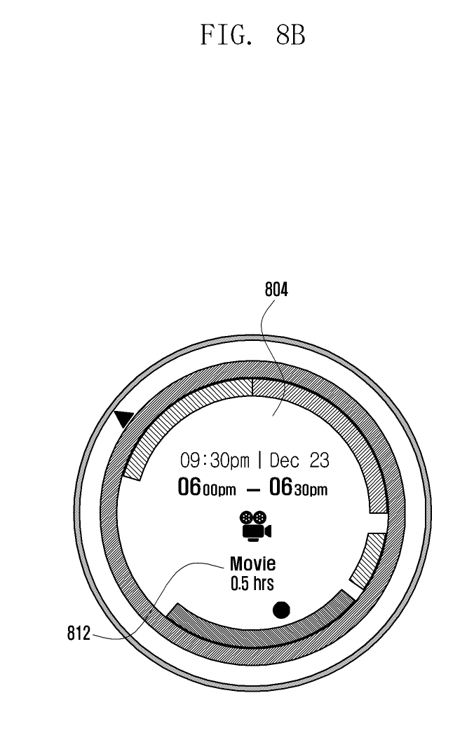

FIG. 8A-8B is a schematic diagram illustrating a scenario in which a user scans through one or more past activities on an electronic device, according to another example embodiment. According to this example embodiment, consider that the user named Rita decides to spend more time on her studies as her exams approach. Accordingly, she has decided not to perform an ‘entertainment’ activity typically performed by her. However, the electronic device may analyze her past behavior pattern, and may recognize that the activity ‘entertainment’ is typically performed for two hours from 6.30 pm to 8.30 pm. Accordingly, in contrast to Rita's intention, the electronic device may add the activity ‘entertainment 802’ as an activity scheduled to be performed on December 23a and indicate the activity ‘entertainment 802’ on a circular UI 804 of the electronic device. The same is illustrated in FIG. 8A.

Thereafter, the electronic device may recognize that Rita has not performed the activity ‘entertainment 802’ scheduled to be performed on December 23 based on the user input or the contexts measured by the sensor unit. Further, the electronic device may recognize that she will miss the activity ‘entertainment 802’ on the basis of her past behavior pattern. Accordingly, the electronic device may recommend performing the activity ‘entertainment 802’ that could not be performed at the regular time in consideration of Rita's schedule. For example, if it is confirmed that there is open time after Rita's exams are over, the electronic device may recommend performance of the past missed activity ‘entertainment 802’ during the open time.

Further, in a certain example embodiment, the electronic device may recommend performance of a sub-activity of ‘entertainment 802’. For example, the electronic device may determine that the open time is insufficient to perform the activity ‘entertainment 802’ in its entirety, and may instead recommend the performance of a sub-activity ‘Movie 812’ for 0.5 hour from 6.00 pm to 6.30 pm next day. This is illustrated in FIG. 8B.

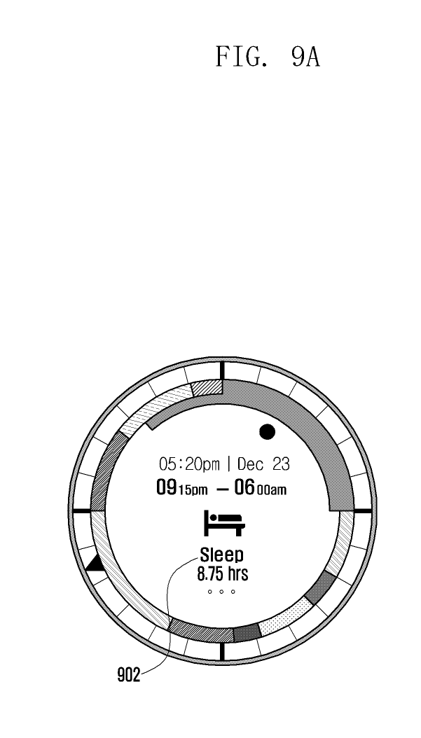

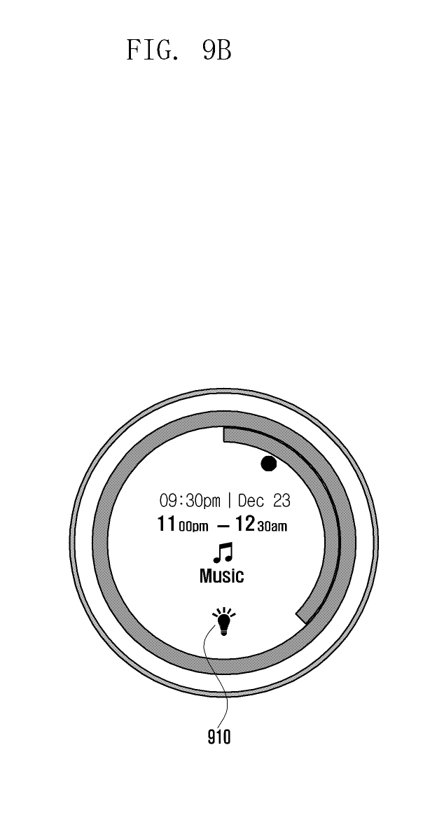

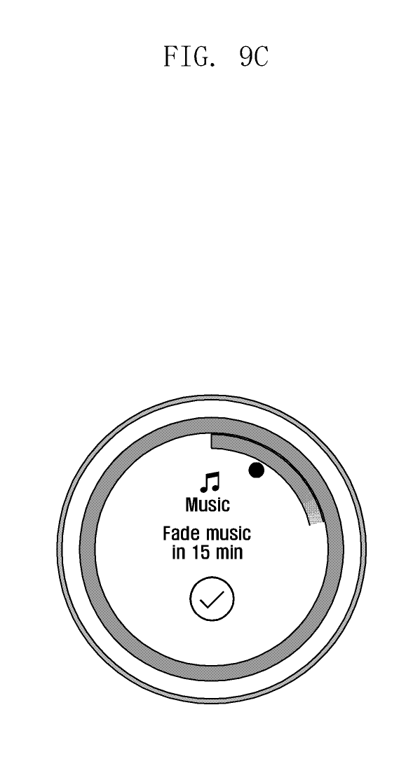

FIGS. 9A-9C are diagrams illustrating a scenario in which a user is scanning through his past activities, according to another example embodiment. According to this example embodiment, a user named, Lee usually likes falling asleep to music. He usually sleeps from 9:15 pm to 6 am. The said activity ‘sleep 902’ is recorded and the same is indicated as first drawing of FIG. 9A. The next day, while checking his watch, Lee realizes that the music was switched on for a long time while he was sleeping. The electronic device identifies the condition and presents an ‘Intelligence button 910’ on the center information space as in FIG. 9B.

Upon switching on ‘Intelligence button 910’, the electronic displays another circular display that allows him to specify the duration after which the music should fade off. Hence, when he goes back to bed to sleep the next time, the music will automatically fade away as per the time he has specified. The same is illustrated in FIG. 9C.

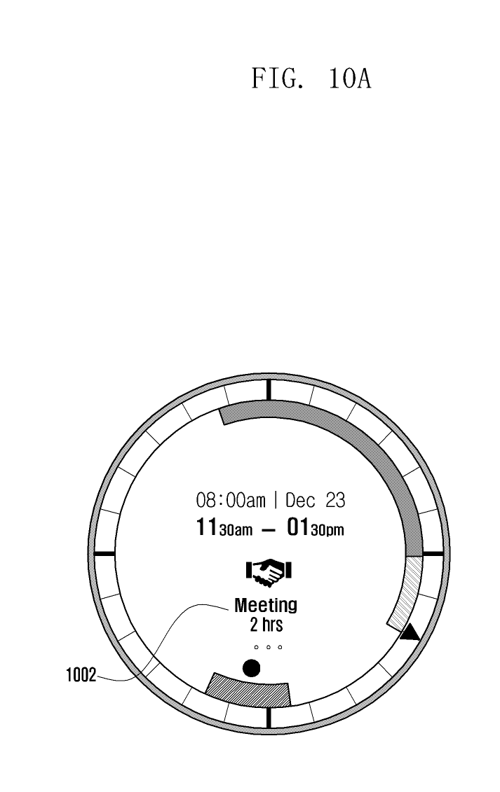

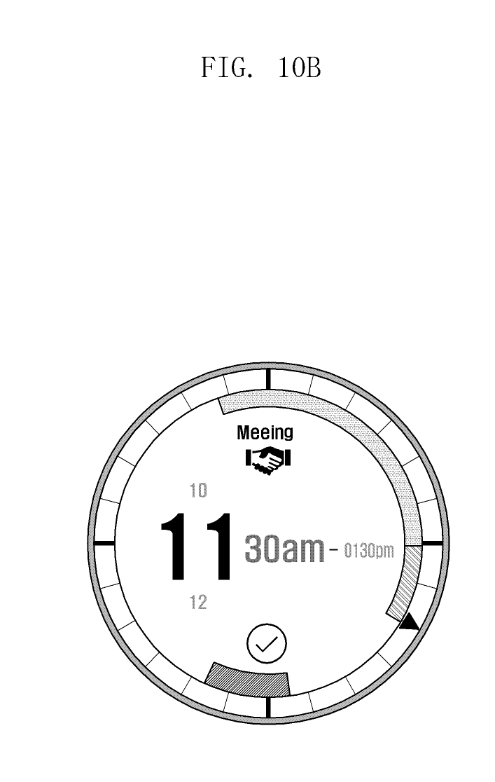

FIG. 10A-10B is a schematic diagram illustrating a scenario in which a user manages his activities, according to one example embodiment. According to this example embodiment, consider that a user has scheduled an event called “meeting 1002” at 11:30 am to 1:30 pm on December 23 as shown in the electronic device of FIG. 10A. In the later part of the day, the meeting was postponed to 2 pm. Therefore, the user needs to create a new entry in the electronic device. The electronic device allows the user to change the timings by providing an edit mode on the circular UI. The same is illustrated in FIG. 10B. Upon editing the time entry, the electronic device will show the meeting to be held at 2 pm.

According to an example embodiment, the user may be scheduled to perform another activity at 4.00 pm. In this case, the electronic device may recommend adjustment of the period of “meeting” scheduled to be performed for two hours. For example, the electronic device may recommend performance of the “meeting” for one and a half hours. Further, in a certain example embodiment, the electronic device may recommend to the user performance of the activity scheduled to be performed at 4.00 later than the scheduled time.

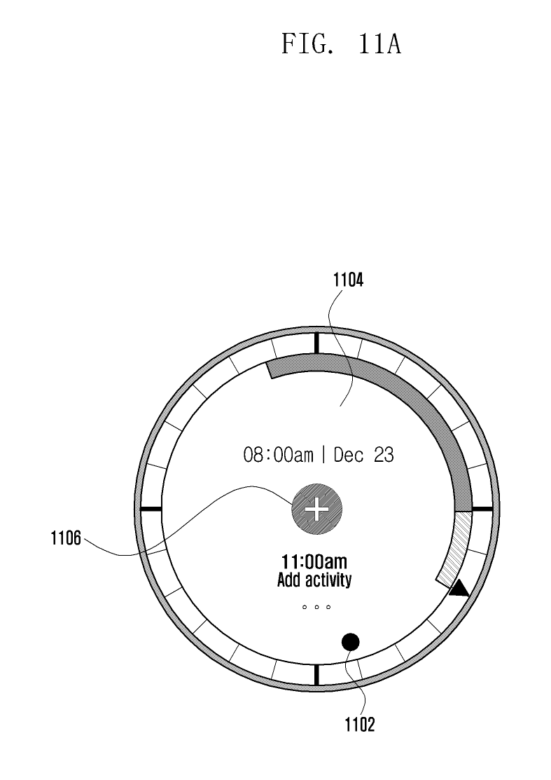

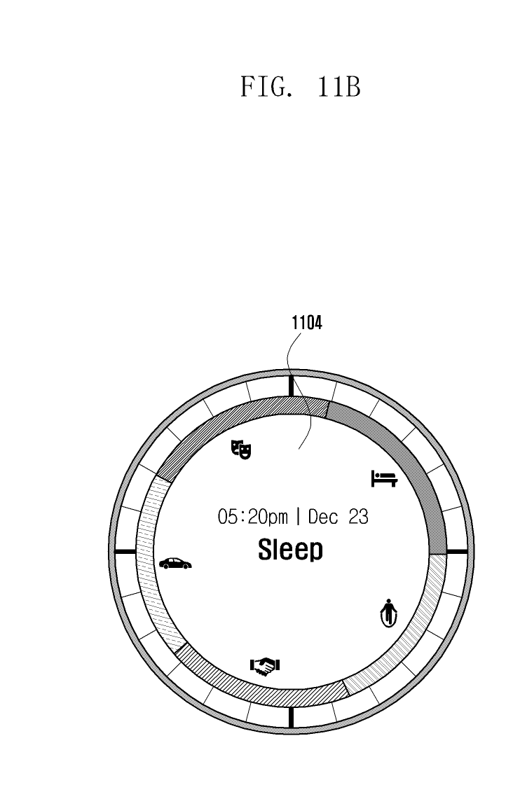

FIGS. 11A-11B are schematic diagrams illustrating a scenario in which a user creates a new activity to be performed in the future, according to one example embodiment. According to this example embodiment, consider that a user wants to add a new activity on her electronic device. To create the new activity, the user has to select an empty space using the activity pointer 1102 on the multi-color coded time chart. In response to the selection, the electronic device displays an ‘Add button 1106’ on the circular UI 1104. The user then adds new activities to her schedule for the day ahead by selecting the ‘Add button 1106’. The newly created activity is illustrated in FIG. 11B.

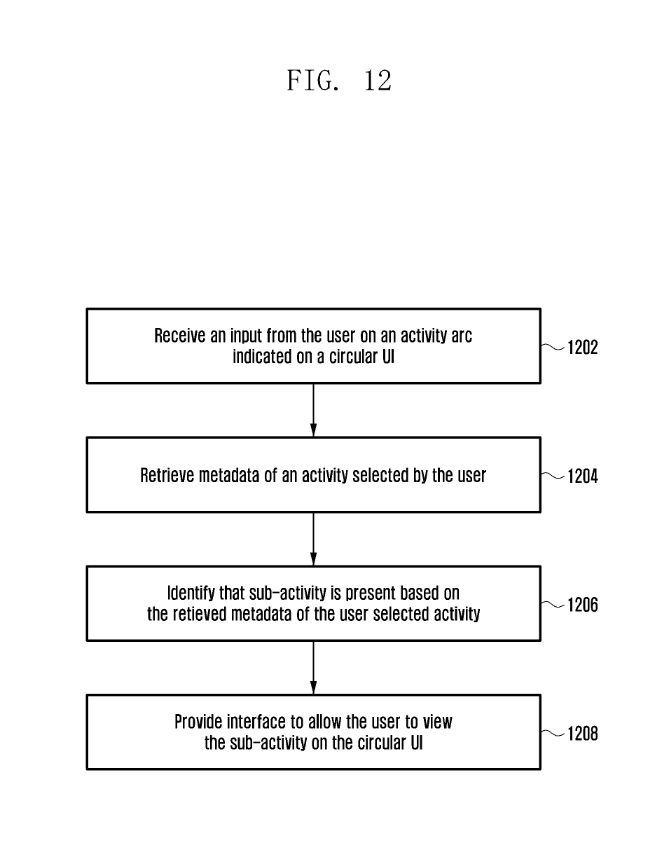

FIG. 12 is a flowchart diagram illustrating an exemplary method of displaying one or more sub-activities associated with a main activity, according to one example embodiment. At step 1202, an input from the user is received on an activity arc indicated on a circular UI. In one example embodiment, the input received provides an instruction to view if there are any sub-activities associated with a main activity. If yes, at step 1204, metadata of an activity is selected by the user. At step 1206, the electronic device identifies a sub-activity based on the retrieved metadata. At step 1208, an interface is provided to allow the user to view the sub-activity on the circular UI.

While the specification has provided detailed descriptions of specific example embodiments, it will be appreciated that those skilled in the art, upon attaining an understanding of the foregoing, may readily conceive of alterations to, variations of, and equivalents to these embodiments. These and other modifications and variations may be practiced by those of ordinary skill in the art, without departing from the scope of the inventive concept. Furthermore, those of ordinary skill in the art will appreciate that the foregoing description is by way of example only, and is not intended to be limiting. Thus, it is intended that the present subject matter covers such modifications and variations.

What is claimed is:

1. An electronic device comprising:
a display;
a sensor unit; and
at least one processor electrically connected to the display and the sensor unit, wherein the processor is configured to:
control the display to display a user interface (UI);
receive an input from a user, via the UI, to display an activity associated with the user;
control the sensor unit to extract context information of the activity associated with the user; and
control the display to display, on the UI, an indication of the activity associated with the user.

2. The electronic device of claim 1, wherein the context information comprises at least one of a time of the activity, a location of the activity, a type of the activity, a duration of the activity, a status of the activity, and behavior and habitual information associated with the user.

3. The electronic device of claim 1, wherein the sensor unit comprises at least one of a biometric sensor, a physiological sensor, an accelerometer, a gyroscope, a polysomnography sensor, a ballistocardiography sensor, a motion sensor, a global positioning system (GPS) receiver, and a proximity sensor.

4. The electronic device of claim 3, wherein the at least one processor is further configured to control the sensor unit to monitor at least one of cardiac activity, hemodynamics, respiratory function, neurological function, body temperature, sleep, stress, toxicity, and the activity of the user.

5. The electronic device of claim 1, wherein the activity comprises at least one of a physical activity and an activity performed on the electronic device by the user.

6. The electronic device of claim 1, wherein the at least one processor is further configured to:
receive an input via a position of the UI;
determine whether the input received on the position of the UI represents open time; and
allow the user to schedule a new activity at the position if the input received represents open time.

7. The electronic device of claim 1, wherein the at least one processor is further configured to:
receive a circular swipe gesture via the UI ;
determine a direction of the circular swipe gesture; and
display one or more future activities on the UI if the circular swipe gesture is provided in a clock wise direction; and
display one or more past activities on the UI if the circular swipe gesture is provided in an anti-clock wise direction.

8. The electronic device of claim 1, wherein the at least one processor is further configured to:
collect information on past activities performed by the user; and
generate user information related to a specific activity based on the information on the past activities; and
provide recommendation information related to the specific activity to the user based on the generated user information.

9. The electronic device of claim 8, wherein the generated user information comprises at least one of a statistical value of the specific activity and user's health information related to performance of the specific activity.

10. The electronic device of claim 8, wherein the recommendation information is related to a generation, a change, or a deletion of the specific activity.

11. A method of tracking a user activity using an electronic device, the method comprising:
displaying a user interface (UI);
receiving an input from a user, via the UI, to display an activity associated with the user;
extracting, by a sensor unit, context information of the activity associated with the user; and
displaying, on the UI, an indication of the activity associated with the user.

12. The method of claim 11, wherein the context information comprises at least one of a time of the activity, a location of the activity, a type of the activity, a duration of the activity, a status of the activity, and behavior and habitual information associated with the user.

13. The method of claim 11, further comprising:
indicating a current time as a pivot point on the UI to scan through past and future activities.

14. The method of claim 11, further comprising:
receiving an input via a position of the UI;
determining whether the input received on the position of the UI represents open time;
allowing the user to schedule a new activity at the position if the input received represents open time.

15. The method of claim 11, further comprising:
receiving a circular swipe gesture via the UI ;
determining a direction of the circular swipe gesture; and
displaying one or more future activities on the UI if the circular swipe gesture is provided in a clock wise direction; and
displaying one or more past activities on the UI if the circular swipe gesture is provided in an anti-clock wise direction.

16. The method of claim 11, wherein the indication is at least one of an icon, text, and a color.

17. The method of claim 11, further comprising:
providing one or more recommendations to the user based on past activity of the user, wherein the one or more recommendations allows the user to perform a current activity.

18. The method of claim 11, further comprising:
collecting information on past activities performed by the user;
generating user information related to a specific activity based on the information on the past activities; and
providing recommendation information related to the specific activity to the user based on the generated user information.

19. The method of claim 18, wherein the generated user information comprises at least one of a statistical value of the specific activity and user's health information related to performance of the specific activity.

20. The method of claim 18, wherein the recommendation information is related to a generation, a change, or a deletion of the specific activity.

21. A wearable electronic device comprising:
a display;
a sensor unit configured to obtain information about a user;
at least one processor electrically connected to the display and the sensor unit, wherein the processor is configured to:
control the display to display a user interface (UI) comprising information regarding a plurality of activities of the user comprising a past activity, a present activity, and a future activity;
control the sensor unit and thereby obtain sensed information about the user;
control a display of at least one of the past activity, the present activity, and the future activity based on the sensed information about the user.
